(19) United States
(12) Patent Application Publication
(10) Pub. No.: US 2024/0082098 A1
(43) Pub. Date: Mar. 14, 2024

(54) STROKE REHABILITATION THERAPY PREDICTIVE ANALYSIS

(71) Applicant: Neurolutions, Inc., Santa Cruz, CA (US)

(72) Inventors: Eric Claude Leuthardt, St. Louis, MO (US); Kern Bhugra, Santa Cruz, CA (US)

(73) Assignee: Neurolutions, Inc., Santa Cruz, CA (US)

(21) Appl. No.: 18/515,755

(22) Filed: Nov. 21, 2023

Related U.S. Application Data
(63) Continuation of application No. PCT/IB2022/054930, filed on May 25, 2022.
(60) Provisional application No. 63/202,116, filed on May 27, 2021.

(51) Int. Cl. A61H 1/02; G06F 3/01

(52) U.S. Cl. CPC A61H 1/0288; G06F 3/015; A61H 2201/1638; A61H 2201/165; A61H 2201/5007; A61H 2201/5061; A61H 2201/5084; A61H 2230/105

(57) ABSTRACT

Methods and systems for assessing a stroke rehabilitation outcome of a subject include a home-based brain-controlled interface (BCI) apparatus and a computer processor in communication with the BCI apparatus. The BCI apparatus has i) a portable brain signal acquisition headset that acquires a brain signal from a subject; ii) an orthosis device having a body part interface configured to be coupled to a body part of the subject and a plurality of sensors that generate force data and movement data; and iii) a BCI component that receives the brain signal from the brain signal acquisition headset. The BCI component is capable of controlling the orthosis device. The computer processor performs instructions to process input data to output a rehabilitation outcome prediction for the subject, where the input data includes the brain signal, the force data, the movement data, and background information about the subject.

### RELATED APPLICATIONS

This application is a continuation of International Patent Application No. PCT/IB2022/054930, filed on May 25, 2022, and entitled “Stroke Rehabilitation Therapy Predictive Analysis”; which claims priority to U.S. Provisional Patent Application No. 63/202,116, filed on May 27, 2021, and entitled “Stroke Rehabilitation Therapy Predictive Analysis”; the contents of which are hereby incorporated by reference in full.

### BACKGROUND

Patients who have had a stroke can experience many types of damage after the event. Effects of a stroke can include issues related to movement of upper and lower extremities of the body such as impaired motor movement, paralysis, pain, weakness, and problems with balance and/or coordination. Other effects of a stroke can include speech impairment, sensory disturbances, memory, and problems with language (aphasia) such as difficulty in speaking, writing, or understanding language.

Rehabilitation programs for motor impairments involve movement therapies to help the patient strengthen muscles and relearn how to perform motions. These movement therapies are often performed by physical therapists and occupational therapists. Brain-computer interface (BCI) systems are another area that have been under development for stroke rehabilitation. In BCI systems, brain signals of a patient are processed and are used to derive intentions of the patient for performing a task. The task can be, for example, moving a cursor on a computer screen or controlling a robotic arm. The task is then implemented using the patient's brain signals through a machine interface such as a robotic mechanism. Ongoing research of BCI systems (which may also be referred to as brain machine interfaces “BMI”) includes studies on identifying factors that affect the ability of a patient to control BCI systems (i.e., BCI-illiteracy), on whether brain signals such as from motor imagery can assist in BCI-based rehabilitation, and comparing the efficacy of BCI rehabilitation to conventional therapy modalities.

### SUMMARY

In embodiments, a system for assessing a stroke rehabilitation outcome of a subject comprises a home-based brain-controlled interface (BCI) apparatus and a computer processor in communication with the BCI apparatus. The BCI apparatus has i) a portable brain signal acquisition headset that acquires a brain signal from a subject; ii) an orthosis device having a body part interface configured to be coupled to a body part of the subject, and having a plurality of sensors that generate force data and movement data; and iii) a BCI component that receives the brain signal from the brain signal acquisition headset. The BCI component is capable of controlling the orthosis device. The computer processor performs instructions to process input data to output a rehabilitation outcome prediction for the subject, wherein the input data includes the brain signal, the force data, the movement data, and background information about the subject.

In embodiments, a method for assessing a stroke rehabilitation outcome of a subject includes collecting a brain signal, force data and movement data from a home-based BCI apparatus. Input data is processed using a computer processor, wherein the input data comprises background information about the subject along with the brain signal, the force data and the movement data to output a rehabilitation outcome prediction for the subject. The home-based BCI apparatus includes a portable brain signal acquisition headset that acquires the brain signal, an orthosis device and a BCI component. The orthosis device has a body part interface configured to be coupled to a body part of the subject and has a plurality of sensors that generate the force data and the movement data. The BCI component receives the brain signal from the brain signal acquisition headset and is capable of controlling of the orthosis device.

### BRIEF DESCRIPTION OF THE DRAWINGS

FIGS. 1A-1B are side views of an orthosis device for a hand, in accordance with some embodiments.

FIGS. 2A-2D are various views of an orthosis device for a hand, in accordance with some embodiments.

FIG. 3A is a general block diagram illustrating a rehabilitation system, in accordance with some embodiments.

FIG. 3B is a schematic of the computer processor of FIG. 3A, in accordance with some embodiments.

FIG. 4 is a block diagram illustrating a system for assessing a stroke rehabilitation outcome of a subject, in accordance with some embodiments.

FIG. 5A is a flowchart of a method for assessing a stroke rehabilitation outcome of a subject, in accordance with some embodiments.

FIG. 5B is a flowchart of a method for training a system to assess a stroke rehabilitation outcome of a subject, in accordance with some embodiments.

FIG. 6 is an isometric view of camera and user interface hardware for a remote rehabilitation assessment, in accordance with some embodiments.

### DETAILED DESCRIPTION

A substantial unmet clinical need exists in chronic stroke patients' access to advanced rehabilitation care and understanding what types of therapies are most effective for a particular patient. For example, although many approaches have been developed for post-stroke motor therapy, approximately 65% of stroke patients with hemiparesis remain unable to use their affected hand six months after their stroke. In the present disclosure, systems and methods are disclosed that can predict the likelihood of rehabilitation success for a stroke patient and identify rehabilitation options best suited for that individual. In particular, embodiments enable predictive analyses that can be performed in a home setting, which can beneficially increase the ability of patients to seek care that is most suitable for their clinical situation. Home-based brain integrated systems are disclosed in which a patient's data is analyzed, such as using cloud processing, in comparison to a stroke population. Outputs include, for example, prediction of a subject's rehabilitation rate and assessment of the subject's rehabilitation progress in terms of a ranking or percentile relative to a general distribution of stroke patients. Embodiments provide a holistic approach by incorporating various factors that may impact a patient's rehabilitation, consequently enabling a medical care team to optimize a treatment plan for that patient.

In this disclosure, embodiments shall be described using an orthosis device for upper extremity therapy as an example—specifically for arm, hand and finger movement. However, embodiments shall also apply to rehabilitation of other body parts of the upper and lower extremities, such as the arm, shoulder, elbow, wrist, hand, leg, knee, ankle or foot. The term “patient” shall be used interchangeably with “subject” as a person being evaluated for stroke rehabilitation.

The present disclosure describes methods and systems for providing in-home assessment after stroke, which can greatly increase the number of stroke patients that can be evaluated and ultimately treated. Furthermore, the ability for patients to perform assessments at home enables the patient's rehabilitation program to be tailored more specifically to their individual needs and progress, thus improving their overall recovery. There is a significant need to create a remote, telehealth approach that can evaluate and ultimately treat chronic stroke patients in the safety of their own home.

Some embodiments enable a predictive analysis involving comparing a patient's data, including underlying factors and BCI performance data, to a larger database and predicting outcomes. Predictive outcomes can serve as a “stroke mass index,” such as whether the patient a good candidate for BCI-based rehabilitation or other therapies, and what the estimated success rate will be. Therapy recommendations can be automatically generated by the system based on, for example, similar patient cohorts or the patient's current progress. In some embodiments, metrics can be utilized to monitor the patient's rehabilitation progress. The monitoring can be used to track or alter the patient's treatment plan and can serve as a motivational tool for the patient. The present systems and methods provide a comprehensive analysis of a patient's outcome by incorporating data that is not conventionally included in BCI systems—such as movement measurements, force measurements, and background information on the patient. The outputs provided by the systems and methods are advantageous in providing composite metrics such as a percentile ranking compared to a broader population with similar conditions, or a score representing their likelihood of success with BCI-based therapy. The systems and methods can also output modified rehabilitation plans and/or alternative therapies that can improve the patient's success rate, by analyzing the various data for that specific individual.

The use of brain-computer interfaces is an emerging technology for post-stroke motor rehabilitation in the chronic setting. BCI technology involves the acquisition and interpretation of brain signals to determine intentions of the person that produced the brain signals and using the determined intentions to carry out intended tasks. BCI technology has been explored in connection with the rehabilitation of impaired body parts, for example, rehabilitation of upper extremity body parts such as arm and hand function impaired due to a stroke event. BCI-mediated stroke therapy allows patients who have motor impairments too severe for traditional therapy to still achieve a functional recovery. BCIs may also be effective for promoting recovery through plasticity by linking disrupted motor signals to intact sensory inputs.

Generally, BCIs do not require patients to generate physical motor outputs. There is a strong premise that BCI-based approaches can be used to develop treatments for patients who are unable to achieve recovery through more traditional methods. Several recent BCI-based treatments in the industry have been shown to aid in motor recovery in chronic stroke patients through varied approaches, such as electrical stimulation or assistive robotic orthoses. BCI-based approaches are thought to drive activity-dependent plasticity by training patients to associate self-generated patterns of brain activity with a desired motor output. Classically, changes in the distribution and organization of neural activity have been identified as a potentially important factor in achieving motor recovery. Motor control is thought to shift to perilesional regions when the primary motor cortex is damaged. Local neural reorganization, however, may not be sufficient for recovery if cortical damage is too severe, or if the ipsilesional corticospinal tract (CST) is substantially transected. Since rehabilitative BCIs often use perilesional or ipsilesional signals, they may not be as effective as rehabilitation systems for patients experiencing high levels of motor impairment. Studies performed in relation to this disclosure have shown that signals acquired from electroencephalogram (EEG) electrodes placed over healthy contralesional motor cortex can be used for BCI control, and that the use of such a system can induce robust functional improvements in chronic stroke patients.

Orthoses in the rehabilitation industry have used various mechanisms to accomplish movement and/or assistance in the movement of impaired body parts. One such mechanism is to physically attach or secure an active movable portion of the orthosis device to the body part that is to be moved or with which movement is to be assisted. The active movable portion of the orthosis device secured to the body part may then be activated to move by a motor or some other form of actuation, and as such accomplish or assist in the movement of the impaired body part secured thereto. Another such mechanism to accomplish or assist in the movement of a body part is through a technique called functional electrical stimulation (“FES”), which involves the application of mild electrical stimuli to muscles that help the muscles move or move better.

Examples of BCI-based systems for use with impaired body parts include descriptions in U.S. Pat. No. 9,730,816 to Leuthardt et al. ('816 patent), under license to the assignee of the present patent application, the content of which is incorporated by reference herein. The '816 patent describes the use of BCI techniques to assist a hemiparetic subject, or in other words, a subject who has suffered a unilateral stroke brain insult and thus has an injury in, or mainly in, one hemisphere of the brain. For that patient, the other hemisphere of the brain may be normal. The '816 patent describes an idea of ipsilateral control, in which brain signals from one side of the brain are adapted to be used, through a BCI training process, to control body functions on the same side of the body.

Additional examples of BCI-based systems for use with impaired body parts include descriptions in U.S. Pat. No. 9,539,118 to Leuthardt et al. ('118 patent), which is commonly assigned with the present patent application and incorporated herein by reference. The '118 patent describes wearable orthosis device designs that operate to move or assist in the movement of impaired body parts, for example body parts that are impaired due to a stroke event, among other conditions described in the '118 patent. For example, the '118 patent describes rehabilitation approaches for impaired fingers, among other body parts including upper as well as lower extremities, using wearable orthosis devices that operate to move or assist in the movement of the impaired body part and that are controlled using BCI techniques. The '118 patent further elaborates BCI-based rehabilitation techniques that utilize brain plasticity to “rewire” the brain to achieve motor control of impaired body parts.

Further examples of BCI-based systems for use with impaired body parts include descriptions in U.S. patent application Ser. No. 17/068,426 ('426 application), which is commonly assigned with the present patent application, and which is incorporated herein by reference. The '426 application describes wearable orthosis device designs that operate to move or assist in the movement of impaired body parts, such as those impaired due to a stroke event, among other conditions described in the '426 application. For example, the '426 application describes an orthosis system that can be operated in one or more of: (i) a BCI mode to move or assist in the movement of the impaired body part based on an intention of the subject determined from an analysis of the brain signals, (ii) a continuous passive mode in which the orthosis system operates to move the impaired body part, and (iii) a volitional mode in which the orthosis system first allows the subject to move or attempt to move the impaired body part in a predefined motion and then operates to move or assist in the predefined motion, such as if the system detects that the impaired body part has not completed the predefined motion.

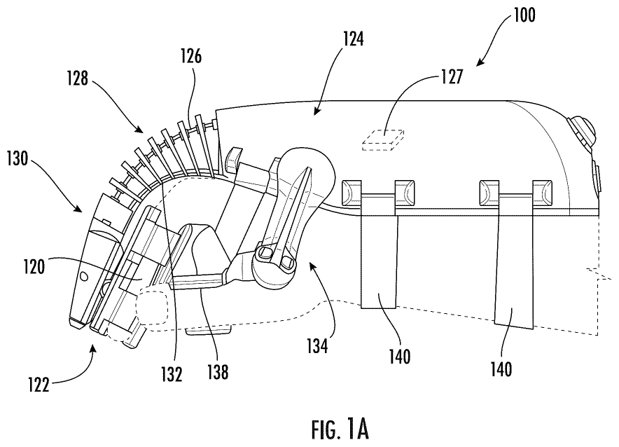

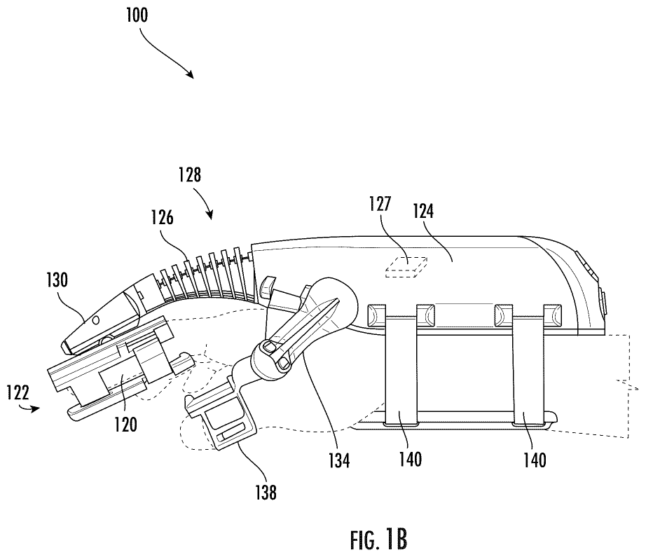

An embodiment of an orthosis device of the '426 application is shown in FIGS. 1A-1B. The orthosis device 100 is shown in a flexed or closed position in FIG. 1A, and in an extended position in FIG. 1B. The wearable orthosis device 100 may receive transmitted signals (for example, wirelessly) containing information about the brain signals acquired by a brain signal acquisition system (e.g., an EEG-based or electrocorticography-based (ECOG) electrodes headset). The orthosis device 100 may then process those received signals to determine intentions using embedded processing equipment, and in accordance with certain detected patient intentions cause or assist the movement of the patient's hand and/or fingers by robotic or motor-drive actuation of the orthosis device 100.

Orthosis device 100 includes a main housing assembly 124 configured to be worn on an upper extremity of the subject. The main housing assembly 124 accommodates straps 140 to removably secure the main housing assembly 124 and thus the other attached components of the orthosis device 100 to the forearm and top of the hand. The straps 140 may be, for example, hook-and-loop type straps. The main housing assembly 124 comprises a motor mechanism configured to actuate movement of a body part of the upper extremity of the subject. A flexible intermediate member 128 (which may also be referred to in this disclosure as a flexible intermediate component or flexible intermediate structure) is configured to flex or extend responsive to actuation by the motor mechanism to cause the orthosis device 100 to flex or extend the secured body part. The wearable orthosis device 100 is designed and adapted to assist in the movement of the patient's fingers, specifically the index finger 120 and the adjacent middle finger (not visible in this view), both of which are securely attached to the orthosis device 100 by a finger stay component 122. The patient's thumb is inserted into thumb stay assembly 134 which includes thumb interface component 138. The main housing structure 124 is designed and configured to be worn on top of, and against, an upper surface (that is, the dorsal side) of the patient's forearm and hand. The finger stay component 122 and thumb stay assembly 134 are body part interfaces secured to the body part (finger or thumb, respectively). A motor-actuated assembly connected to the body part interface moves the body part interface to cause flexion or extension movement of the body part.

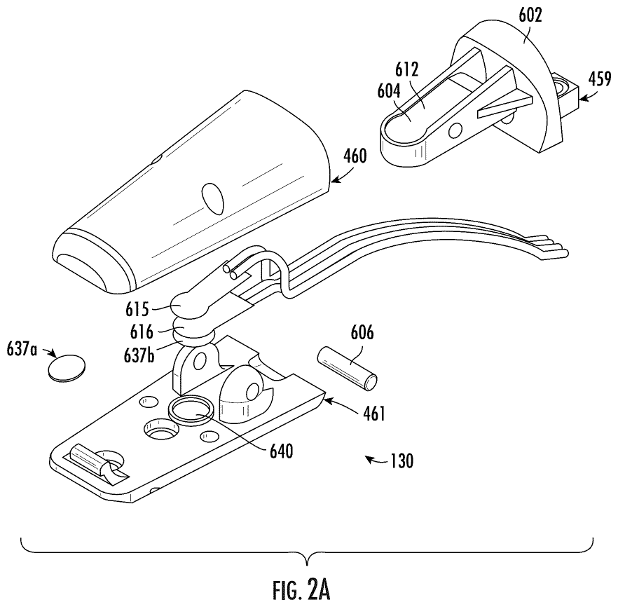

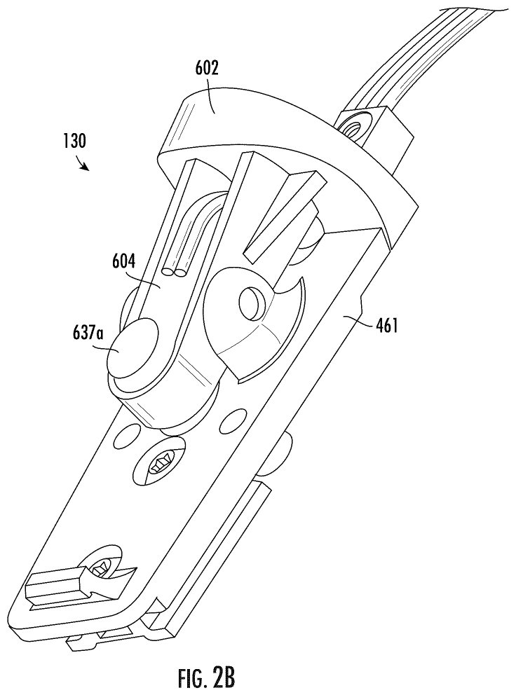

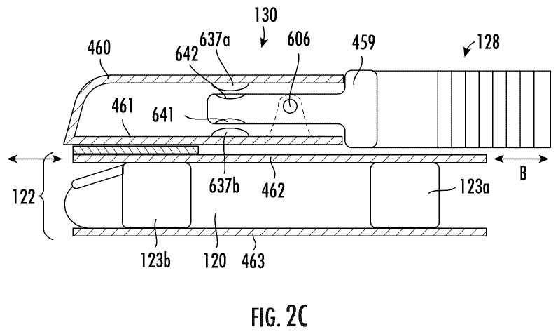

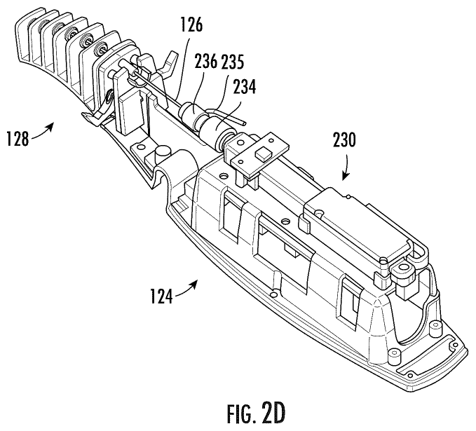

The motor-actuated assembly may be configured as a linear motor device (e.g., motor mechanism 230 as shall be described in FIG. 2D) inside the main housing structure 124. The linear motor device longitudinally advances and retracts a pushing-and-pulling wire 126 that extends distally from the distal end of the main housing structure 124 and extends longitudinally through the flexible intermediate structure 128 and connects to a connection point on a force sensing module (“FSM”) 130. The flexible intermediate component 128 has a flexible baffle structure. When a linear motor in the main housing structure pulls the wire 126 proximally, the attached FSM 130 is pulled proximally. This motion causes the flexible intermediate structure 128 to flex so its distal end is directed more upwardly, causing or assisting in extension movement of the secured index and adjacent middle fingers. The upward flexing of the flexible intermediate structure 128 so that its distal end is directed more upwardly (and also its return) is enabled by the baffle structure of the flexible intermediate structure 128. In particular, a generally flat bottom structure 132 is provided on the flexible intermediate structure 128, where the bottom structure 132 is configured to attach to a bottom or hand-side of each of the individual baffle members. The opposite or top-side of each of the individual baffle members are not so constrained and thus are free to be compressed closer together or expanded further apart by operation of the pushing-and-pulling wire 126 enlarging and/or reducing the top-side distance between the distal end of the main housing structure 124 and the proximal end of the FSM 130.

The FSM 130 serves a force sensing purpose, comprising force sensors that are capable of measuring forces caused by patient-induced finger flexion and extension vis-à-vis motor activated movements of the orthosis device 100. In embodiments, the force sensors measure force data of forces applied between the body part interface and the motor-actuated assembly. The force sensing function of the FSM 130 may be used, for example, to ascertain the degree of flexion and extension ability the patient has without assistance from the orthosis device 100, to determine the degree of motor-activated assistance needed or desired to cause flexion and extension of the fingers during an exercise, or other purposes.

In embodiments of this disclosure, electro-mechanical orthosis devices are used to acquire significant amounts of meaningful data about a patient's clinical performance, including monitoring utilization data, open/close success rates, force profile characteristics, accelerometer info as well as motor position metrics related to range of motion. In embodiments, the orthosis device has a body part interface configured to be coupled to a body part of the subject and has a plurality of sensors that generate force data and movement data. The plurality of sensors may include one or more of a force sensor, an accelerometer or a gyroscope. For example, force sensors in the FSM 130 can measure passive hand opening force (spasticity), active grip strength and extension force. Housing 124 may contain a six-axis inertial measurement unit (IMU) having an accelerometer and gyroscope to monitor motion sensing, orientation, gestures, free-fall, and activity/inactivity. A motor potentiometer to measure position for facilitating the evaluation of the range of motion may also be included in either the FSM 130 or housing 124. The accelerometer, gyroscope and potentiometer are examples of movement sensors that are represented by movement sensor 127. The orthosis device 100 has substantial sensor and mechanical capabilities to physically interact with the limb and hand of a stroke patient which can be leveraged to provide remote functional metrics comparable to portions of those performed in a conventional in-person motor assessment such as a Fugl-Meyer (FM) evaluation.

FIGS. 2A-2D show details of sensors in the FSM 130 of orthosis device 100, in accordance with some embodiments. Further description of these sensors may be found in the '426 application, incorporated by reference above. In FIGS. 2A-2B, a vertically oriented proximal end plate 602 has an elongate extension component 604 extending distally from the end plate 602. The elongate extension component 604 serves as a carrier of two force sensing resistors 615, 616. A horizontally oriented dividing wall 612 of the extension component 604 separates the structure of the two force sensing resistors (“FSRs”) 615, 616 from one another, or in other words, separates a first FSR 615 that may be assembled to be located above the dividing wall 612 (hereafter called the “top” FSR 615) from a second FSR 616 that may be assembled to be located below the dividing wall 612 (hereafter called the “bottom” FSR 616).

To provide the force sensing capability of the connecting/FSM assembly 130, two force sense resistor (“FSR”) bumpers, buttons, or plungers 637a, 637b are utilized. A first FSR bumper 637a is fixedly positioned on an underside surface of an upper shell 460 of FSM 130 in a location thereon aligned with the top FSR 615, so that the top FSR's upwardly facing surface (that is, its force sensing surface, labeled as 642 in FIG. 2C) comes in contact with and bears upon the first FSR bumper 637a when a distal end of the central support 459 rocks or pivots upwardly relative to the fixed-together upper and lower shells 460, 461. A second FSR bumper 637b is fixedly positioned onto and within an opening or recess 640 provided on a top surface of the lower shell 461 in a location thereon aligned with the bottom FSR 616, so that the bottom FSR's downwardly facing surface (that is, its force sensing surface, labeled as 641 in FIG. 2C) comes in contact with and bears upon the second FSR bumper 637b when a distal end of the central support 459 rocks or pivots downwardly relative to the fixed-together upper and lower shells 460, 461.

When the distal end of the central support 459 rocks or pivots downwardly relative to the upper and lower shells 460, 461, the force sensing surface 642 of first FSR 615 may become no longer in contact with the first bumper 637a; and when the distal end of the central support 459 rocks or pivots upwardly relative to the upper and lower shells 460, 461, the force sensing surface 641 of second FSR 616 may become no longer in contact with the second bumper 637b. The rocking or pivoting of the central support 459 may be limited by constraints imposed by the clearances of the two bumpers 637a, 637b from their respective FSRs 615, 616. In some embodiments, such clearances are minimized so that the amount of rocking or pivoting permitted is minimized but the force-sensing functioning of both FSRs is still enabled.

FIG. 2C is a side cross-sectional view illustrating that finger stay component 122 is slidably engaged with the underside of the lower shell 461. This sliding engagement is illustrated by arrow B. Accordingly, the lower shell 461 of the connecting/FSM assembly 130 is connected to the finger stay component 122 that is attached thereunder in a manner that the angular orientation of the FSM 130 and the finger stay component 122 remain fixed, and yet the finger stay component 122 is permitted to freely move or slide longitudinally with respect to the lower shell 461. The finger stay component 122 is provided with an upper plate 462 that rests above the two secured fingers 120 and a lower generally horizontal plate 463 that rests below the two fingers. Two adjustable straps 123a, 123b are provided with the two plates 462, 463 to secure the index and middle fingers as a unit between the two plates.

A discussion of how these force sensing capabilities may be utilized in an orthosis device shall be described in reference to FIG. 2C. In a first example, the orthosis device is not being actuated but that the patient is opening/extending his or her fingers under his or her own force. The orthosis device is able to be “forced” open (that is, forced into an “extended” position) by the patient's own finger opening force, which in some cases may involve activating a motor associated with orthosis device to be enabled to “follow” the volitional action of the subject. In other words, although it is the patient's own finger operating force that induces such movement in the orthosis device, the linear actuator may be “turned on” to allow the fingers to open with the patient's own force (without assist). The patient's own finger opening force causes a portion of the lower shell 461 distal of the pivot point/dowel 606, including the bottom bumper 637b affixed thereto, to be moved upwardly relative to portion of the central support that is also distal of the pivot point/dowel 606, such that the dome surface of the bottom bumper 637b contacts and applies a force against the downward facing sensing surface 641 of the bottom FSR 616. As such, the bottom FSR 616 captures a measurement from which the patient's finger opening force may be determined.

In a second scenario, the patient is closing/flexing his or her fingers under his or her own volition and the orthosis device again is not being actuated but is able to “follow” the subject's volitional action so that the orthosis device may be “forced” into a flexed or closed position by the patient's own finger closing force. In this second scenario, the patient's own finger closing force causes a portion of the upper shell 460 that is distal of the pivot point/dowel 606, and thus the top bumper 637a affixed thereto, to be “pulled” downwardly such that the domed surface of the top bumper 637a is put in contact with and applies a force against the upwardly facing sensing surface 642 of the top FSR 615. As such, the top FSR 615 enables measurement of a patient's “finger closing force.”

In another scenario, the orthosis device is actuated to open/extend the finger stay component 122 and hence open/extend the patient's fingers secured thereto, but the patient is not able to provide any finger opening/extension force. In this case, the flexible intermediate structure 128 may be actuated so that its distal end is oriented more upwardly to move the connecting/FSM assembly's central support 459 upwardly and in a clockwise direction. Because in this scenario it is assumed that the patient will be providing no help in opening the fingers, a distal portion of the upper and lower shells 460, 461 will “rock” downwardly in a counter-clockwise direction relative to the central support 459 so that the upwardly facing sensing surface 642 of the top FSR 615 comes in contact with and bears against the top bumper 637a affixed to the inner surface of the upper shell 460. In this case, the downwardly facing sensing surface 641 of the bottom FSR 616 will no longer be in contact with the bottom bumper 637b affixed to the lower shell 461. In this scenario, the presence of a force at the top FSR 615 and absence of a force at the bottom FSR 616 may thereby inform the orthosis device that the patient is providing little or no assistance in the finger opening/extension movement that is being actuated by the orthosis device.

In an opposite scenario, the orthosis device is actuated again, this time to close or flex the finger stay component 122 and hence close or flex the patient's fingers. In this scenario, the patient is not able to provide any finger closing or flexing force, but instead will be moved into a flexed position by operation of the orthosis device. In this case, the flexible intermediate structure 128 is actuated so that its distal end becomes oriented more downwardly, which in turn causes the connecting/FSM assembly's central support 459 to be moved downwardly in a counterclockwise direction. Because in this scenario the patient is providing no help in closing the fingers, the fixed-together upper and lower shells 460, 461—which again are in a fixed angular orientation with respect to the finger stay component 122 and hence to the patient's fingers—will then “rock” in a clockwise direction relative to the central support 459 until the downwardly facing sensing surface 641 of the bottom FSR 616 comes into contact with and bears against the bottom bumper 637b affixed to the lower shell 461. In addition, the upwardly facing sensing surface 642 of the top FSR 615 will then be free of contact with the top bumper 637a affixed to the upper shell 460. In this scenario, the presence of a force at the bottom FSR 616 and absence of a force at the top FSR 615 may thereby inform the orthosis device that the patient is not providing any assistance in the finger closing/flexing movement that is being actuated by the orthosis device.

In yet another scenario, the orthosis device is actuated to open/extend the finger stay component 122, but the patient is providing a full finger opening force beyond the opening/extension force being provided by the orthosis device. In this scenario, despite the fact that the flexible intermediate structure 128 is providing a force that would move the central support 459 upwardly, the patient is providing an additional opening/extending force on the finger stay component 122 and thus on the upper and lower shells 460, 461 angularly affixed thereto, and as such, the patient is volitionally causing the upper and lower shells 460, 461 to move at even faster rate than the actuated central support 459 is being actuated by the orthosis device. As such in this scenario, the bottom bumper 637b affixed to the lower shell 461 may come in contact with and bear against the bottom FSR's downward facing sensing surface 641, and the top bumper 637a affixed to the upper shell 460 may then be free of and thus provide no force against the top FSR's upward facing sensing surface 642. As such, in this scenario the presence of a force sensed at the bottom FSR 616, and absence of a force sensed at the top FSR 615 may inform the orthosis device that the patient is providing all the necessary finger opening force to achieve the desired finger opening/extending.

In other implementations, load cell force sensing may be used in connection with the pushing-and-pulling wire 126 (FIGS. 1A-1B), to provide for the above-described force sensing capabilities. In one implementation shown in FIG. 2D, a sensor 234 and a coupler 236 are mounted on an end of a motor mechanism 230 that includes a motor and a linear actuator. The motor mechanism 230 may be referred to in this disclosure as a motor-actuated assembly. The sensor 234 may be, for example, a load cell with wiring 235. Other types of sensors (for sensor 234 or elsewhere within the orthosis device) may be used such as position sensors (e.g., optical, proximity), other force sensors (e.g., strain gauge, pressure sensor), and limit switches. The coupler 236 receives and holds the pushing-and-pulling wire 126. The load cell force sensor 234 is in the form of a cylindrical drum-shaped structure and may be provided in series with the pushing-and-pulling wire 126, for example, with one side of the drum-shaped structure facing proximally and the opposite side of the drum-shaped structure facing distally. In this implementation, the pushing-and-pulling wire 126 may comprise two portions of wire, a proximal portion of wire 126 and a distal portion of wire 126. The proximal portion of the pushing-and-pulling wire 126 may have its proximal end attached to a distal end of a linear motor inside the main housing structure 124, and its distal end fixedly attached to a proximally facing side of the load cell drum shaped structure. The distal portion of the pushing-and-pulling wire 126 may have its proximal end fixedly attached to a distally facing side of the load cell drum-shaped structure and its distal end fixedly attached to the force sensing module 130 (FIGS. 2A-2C).

A load cell force sensor design may be selected that is capable of sensing both a tension force (exerted on the load cell force sensor 234, for example, by a pushing-and-pulling wire 126 being extended distally against the load cell force sensor) and a compression force (exerted on the load cell force sensor, for example, by a pushing-and-pulling wire being pulled proximally to effectively “pull” on the load cell force sensor). Accordingly, such an implementation of a force sensing module may provide functionality in connection with a volitional mode of operation of the orthosis device.

In embodiments for assessing or predicting rehabilitative outcomes for a patient, force sensors in the orthosis device may include one or more of the force sensing resistors 615, 616; the force sense resistor bumpers 637a, 637b; or load cell force sensor 234.

Other types of sensors may be included in orthosis devices that are used for the rehabilitative assessments of the present disclosure. For example, movement sensors such as accelerometers, gyroscopes, and/or potentiometers may be used to measure position, speed, acceleration, and/or orientation. Furthermore, any of the sensors described in this disclosure may be used in orthosis devices of types other than that shown in FIGS. 1A-1B and 2A-2D. For example, embodiments may utilize orthosis devices for other movements of the upper extremity such as the elbow or shoulder. In other examples, embodiments may utilize orthosis devices for the lower extremity, where sensors may be used to detect forces and movement of the hip, knee, ankle, foot, and toes.

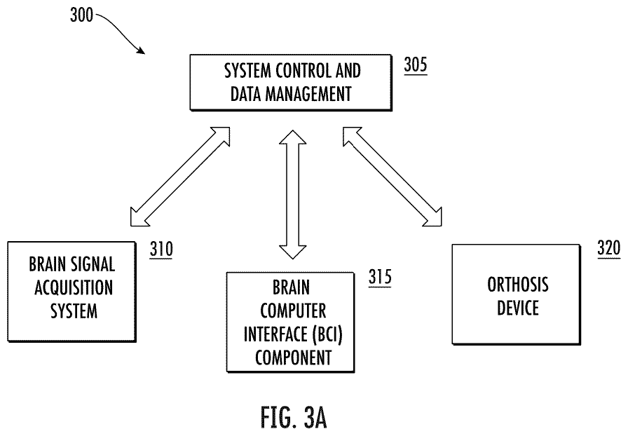

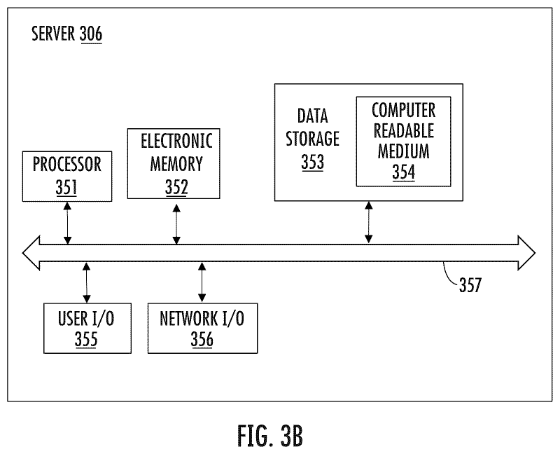

FIG. 3A shows a generalized block diagram of a rehabilitation system 300 using a BCI-based orthosis device such as the device of FIGS. 1A-1B and 2A-2D. The rehabilitation system 300 may be used for movement of various body parts, for example, an arm, shoulder, elbow, wrist, hand, finger, leg, knee, ankle, foot, or toe. As shown in FIG. 3A, the rehabilitation system 300 includes: (i) a system control and data management component or components 305; (ii) a brain signal acquisition system 310; (iii) a brain computer interface (BCI) component 315; and (iv) an orthosis device 320. The orthosis device 320 may be a body-worn, and thus a portable, body part movement control and/or movement assistance system. The system control and data management component 305 may be configured as one or more computer processors. The system control and data management component 305 may include not only local control and data management of the rehabilitation system 300, namely, at a site co-located with a subject performing a BCI session (and perhaps integrated with the BCI component 315 and/or the orthosis device 320 or integrated in a local computing device such as a tablet computer), but also may include a remote, network accessible central rehabilitation management computing system. A central rehabilitation management computing system may be used, for example, in set-up and on-going operation of the system, and may be located at a location that is remote of the patient, for example, at a healthcare facility or the facilities of some other type of services provider.

FIG. 3B is a simplified schematic diagram showing an embodiment of a server 306 (representing any combination of one or more servers, i.e., computer processors) for use as the system control and data management component 305 of the rehabilitation system 300, in accordance with some embodiments. Other embodiments may use other components and combinations of components. For example, the server 306 may represent one or more physical computing devices (e.g., computer hardware processors) that include or are in communication with computer servers, such as web servers, rack-mounted computers, network storage devices, desktop computers, laptop/notebook computers, etc., depending on the complexity of the system. In some embodiments implemented at least partially in a cloud network potentially with data synchronized across multiple geolocations, the server 306 may include one or more cloud servers. In some embodiments, the functions of the server 306 are enabled in a single computer device. In more complex implementations, some of the functions of the computing system are distributed across multiple computing devices, whether within a single computer processor, a single server farm facility or multiple physical locations.

In the illustrated embodiment, the server 306 generally includes at least one processor 351, a main electronic memory 352, a data storage 353, a user input/output (I/O) 355, and a network I/O 356, among other components not shown for simplicity. The components are connected or coupled together by a data communication subsystem 357. A non-transitory computer readable medium 354 includes instructions that, when executed by the processor 351, cause the processor 351 to perform operations including processing input data for the predictive analysis of rehabilitation as described herein. The instructions may include machine learning algorithms.

In accordance with the description herein, the various components of the presents systems or methods generally represent appropriate hardware and software components for providing the described resources and performing the described functions. The hardware generally includes any appropriate number and combination of computing devices, network communication devices, and peripheral components connected together, including various processors, computer memory (including transitory and non-transitory media), input/output devices, user interface devices, communication adapters, communication channels, etc. The software generally includes any appropriate number and combination of conventional and specially developed software with computer-readable instructions stored by the computer memory in non-transitory computer-readable or machine-readable media and executed by the various processors to perform the functions described herein.

Returning to FIG. 3A, generally, the brain signal acquisition system 310 acquires brain signals, performs low-level signal processing, and transmits the brain signals, for receipt by the BCI component 315 under control of the system control and data management component 305. The brain signals may be acquired by the acquisition system 310 using a number of arranged electrodes that are part of the acquisition system. These electrodes may be EEG surface electrodes or implantable electrodes. For example, brain signal acquisition system 310 may use electrodes on the surface of the scalp (e.g., dry electrodes, wet electrodes), or implantable electrodes (e.g., ECOG electrodes on the brain's surface or “point-style” where only the electrode tips penetrate into brain tissue). The acquired neural signals may include magneto encephalography (MEG) signals, mu rhythm signals, beta rhythm signals, low gamma rhythm signals, high gamma rhythm signals, action potential firing, and the like. The brain signal acquisition system 310 may also include processing circuitry to perform the low-level processing and formatting of brain signal information for use by the BCI component 315, as well as a connection interface to enable that transmission. The connection for transmission between the brain signal acquisition system 310 and the BCI component may be wireless or hard-wired and may be direct or indirect through intermediate components, and thus a connection interface in the brain signal acquisition system 310 and the components with which the system 310 communicates would be adapted accordingly to enable the wireless or hard-wired transmissions. For example, a connection interface may include USB interface devices, BLUETOOTH® communication devices, Wifi communication devices or some other wireless or hard-wired transmission protocol interface mechanisms and circuitry.

In some implementations, body-worn equipment of the system 300 may include both the movable and actuatable equipment to cause body parts to be moved or assist in their movement as well as the BCI component 315. The BCI component 315 in this example may generally include BCI processing capability that is adapted to be worn on a user (e.g., on the user's forearm as in the FIG. 1 example or some other body part in other implementations). The body movement assistance component in such an implementation may be operably connected to the BCI component 315, and also may be adapted to be worn by the user (e.g., on a user's hand as in the FIG. 1 example or some other body part to be moved in other implementations).

The system 300 may include various components for providing information to and receiving input from a user. Visual output display equipment, for example, may be a regular or touch screen display for providing visual prompts (e.g., graphics, instructions, etc.) or other sorts of information to the user and/or for receiving user input. The input devices, for example, may include one or more buttons for controlling (e.g., pausing, powering on/off, sending data, receiving data, changing modes, etc.) the wearable device. For example, input devices such as buttons may serve as soft keys alongside display equipment and/or may be situated away from the display equipment. Audio output equipment (e.g., speakers), for example, may be used for providing auditory prompts (e.g., live or recorded spoken instructions, tones indicating success or error conditions, etc.). Audio input equipment (e.g., microphone), for example, may be used for receiving spoken input from the user (e.g., voice controls) and/or may serve with the audio output equipment for conducting a live communication session with a remote technician.

In terms of software and/or firmware programs, the system control and data management system 305 and BCI component 315 may include various programs that are stored in non-volatile memory that include executable program instructions that are executed by a CPU to carry out the various processing functions. This may include one or more of the following program modules: (i) a neural signal interpreter for interpreting neural signals received from the brain signal acquisition system 310, and specifically determine whether those received signals are indicative of a user intention to perform certain predefined body movements which will be caused or assisted by the orthosis device 320; (ii) a device control module for providing control signals to the orthosis device to actuate movement; (iii) a training mode module for carrying out training processes; (iv) an operational mode module for carrying out the operation of the system 300 in normal operation, for example, in a rehabilitation session, (v) a calibration mode module for carrying out the operations calibration processes, and (vi) a communications module for carrying out communications processes between the brain signal acquisition system 310, the BCI component 315, and the orthosis device 320, and a central network-accessible rehabilitation management system.

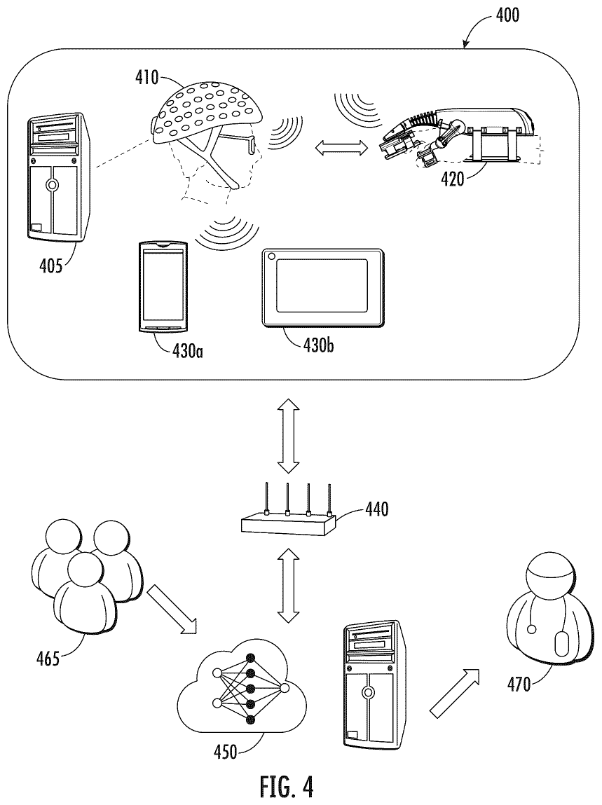

FIG. 4 is a schematic diagram of a system 400 for assessing a stroke rehabilitation outcome of a subject, in accordance with some embodiments. System 400 is a home-based brain-controlled interface system that includes a portable brain signal acquisition headset 410 (serving as the brain signal acquisition system 310 of FIG. 3) that acquires brain signals from a subject, an orthosis device 420 (equivalent to orthosis device 320 of FIG. 3), and a control system 405 configured to operate the orthosis device 420. The home-based system 400 is sized to be portable and used in a patient's personal home, in contrast to institutional-type BCI equipment that are room-sized and therefore suited for medical facilities. The brain signal acquisition headset 410 and orthosis device 420 are small enough in dimension and lightweight enough for a patient to carry, use and store the equipment in their home. The home-based system 400 can be used by a patient at a location remote from a medical facility and without the need for a medical professional to set up or operate the system. A computing device such as a smartphone 430a or tablet computer 430b may also be included in system 400 to enable input of information from the subject and to display results for the subject to view. The headset 410, orthosis device 420, and computing devices (430a or 430b) are all in communication with each other, such as wirelessly. For example, an application program provided on the computing device 430a,b may communicate with the EEG brain signal acquisition headset 410 through wireless communications using a protocol such as Bluetooth, and with the orthosis device 420 through wireless communications using a protocol such as Wifi Direct.

A BCI component 315 (not shown in FIG. 4) receives the brain signals from the brain signal acquisition headset 410 and is capable of controlling of a body part interface of the orthosis device 420. The BCI component—which can be provided in the wearable orthosis device 420, computing device (e.g., smartphone 430a or tablet computer 430b), or in headset 410—processes the brain signals to determine the patient's intentions. Control system 405 is configured to operate the orthosis device 420 in three modes: 1) a BCI mode in which the orthosis device operates to move or assist in the movement of the body part based on an intention of the subject determined from an analysis of the brain signals; 2) a continuous passive mode in which the orthosis device operates to move the body part with no volition from the subject; and 3) a volitional mode in which the orthosis device first allows the subject to move or attempt to move the impaired body part in a predefined motion and then operates to move or assist in the predefined motion of the body part.

Also shown in FIG. 4 is a router 440 which sends data from home-based system 400 to a computer processor 450, such as through wireless communications using a WiFi protocol. Computer processor 450 may be a cloud-based system (configured, e.g., as described for the server 306 of FIG. 3B) and includes a memory containing instructions, which may incorporate a machine learning algorithm, to process input data. The input data includes information from home-based system 400 as well as data from a larger stroke population database 465. The computer processor 450 outputs a predictive rehabilitation outcome which is then sent back to the patient via router 440 and to a care team 470 which may be the patient's physician, medical care team or other third party.

The system 400 for assessing a stroke rehabilitation outcome of a subject can include a home-based brain-controlled interface apparatus and a computer processor in communication with the BCI apparatus. The computer processor operates according to instructions to process input data to output a rehabilitation outcome prediction for the subject. In some embodiments, the computer processor is supplied separately from the system 400, where the system 400 has a connection for connecting to the computer processor for processing the data collected from the BCI apparatus. The home-based BCI apparatus includes: i) a portable brain signal acquisition headset that acquires a brain signal from a subject, ii) an orthosis device, iii) a BCI component that receives the brain signals from the brain signal acquisition headset and that is capable of controlling of the body part interface, and iv) a control system. The orthosis device includes a body part interface configured to be coupled to a body part of the subject, and a plurality of sensors that generate force data and movement data. The plurality of sensors may comprise a force sensor that generates the force data, where the force data includes forces applied between the body part interface and the motor-actuated assembly. In some embodiments the orthosis device includes a motor-actuated assembly that actuates the body part interface to move the body part; a force sensor that measures force data applied between body part interface and the motor-actuated assembly; and a movement sensor that measures movement data of the body part interface. The control system is configured to operate the orthosis device in three modes: a BCI mode in which the orthosis device operates to move or assist in the movement of the body part based on an intention of the subject determined from an analysis of the brain signal; a continuous passive mode in which the orthosis device operates to move the body part with no volition from the subject; and a volitional mode in which the orthosis device first allows the subject to move or attempt to move the impaired body part in a predefined motion and then operates to move or assist in the predefined motion of the body part. The computer processor is in communication with the BCI apparatus, the computer processor performing instructions to process input data to output a rehabilitation outcome prediction for the subject, wherein the input data includes the brain signals, the force data, the movement data, and background information about the subject.

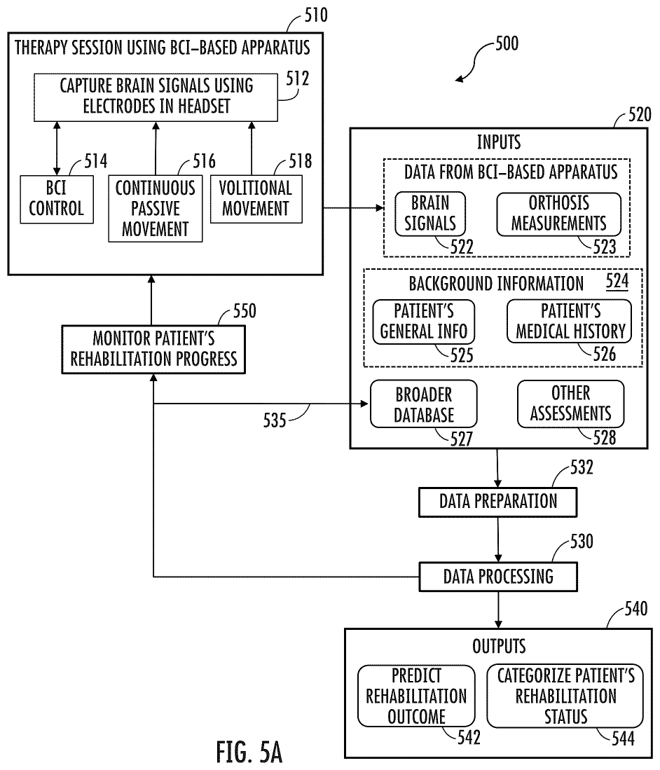

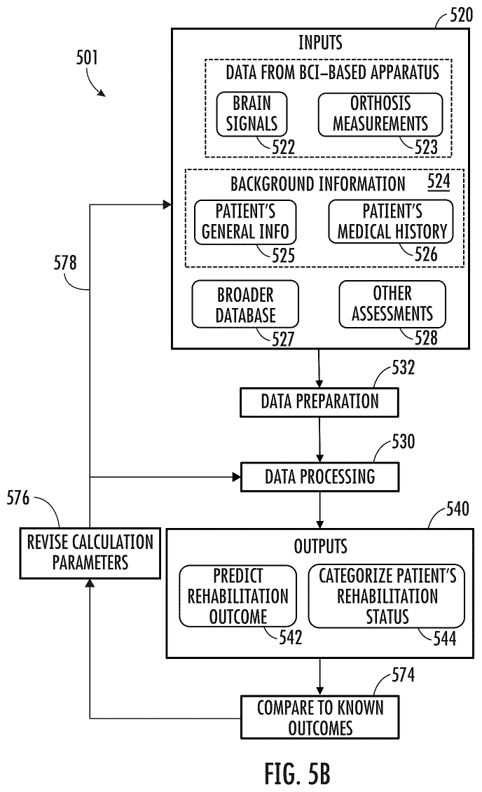

The home-based system 400 has several features that enable it to uniquely provide meaningful and more comprehensive rehabilitation outcome predictions and assessments than could be achieved with conventional orthoses devices. First, the headset 410 is portable, enabling a subject to collect EEG or other brain signal information in their home. This home-based system can facilitate more frequent measurement sessions compared to the subject having to go to a medical facility each time an assessment is to be performed. Another beneficial feature of home-based system 400 is that multiple modes are available in which either the user can control the orthosis device (see block 514 in FIG. 5A) or the robotic device is actuated by its control system to then generate brain signals in the subject (blocks 516 and 518 of FIG. 5A). Consequently, more detailed data on the status of neural pathways between a body part and the brain can be understood compared to conventional orthoses. The three operation modes of the system 400—BCI (resting state), CPM (passive movement), and volitional (active movement)—are unique compared to conventional orthosis systems. Furthermore, home-based system 400 utilizes proportional proprioceptive input in that the user can reliably control the amplitude of the brain wave, which uniquely provides more meaningful data than conventional orthosis devices. Other benefits of home-based system 400 which result in more meaningful data than conventional systems include high cortical representation (e.g., hand movement involves a large amount of the cortex) and ipsilateral control.

FIG. 5A is a flowchart of a method 500 for assessing a stroke rehabilitation outcome of a subject, in accordance with some embodiments. In block 510, a therapy session is performed using a BCI-based apparatus. Block 510 involves collecting brain signals, force data and movement data from a home-based brain-controlled interface apparatus such as home-based system 400 of FIG. 4. The therapy session involves capturing brain signals in block 512 using electrodes in a signal acquisition headset, such as an EEG, ECOG, or other electrophysiology measurement devices as described in this disclosure. The headset may include a plurality of sensors, such as four, six, or more than six sensors. The patient wears an orthosis device, such as described in FIGS. 1A-1B and 2A-2D, that is in communication with the brain signal acquisition headset. In a BCI control mode in block 514, patient-generated brain signals are used to determine the intention of the patient, and then the orthosis device is operated to execute those motions based on the intentions. In other words, in BCI control mode, the electrical signals that control movement of the orthosis device are internally induced (i.e., by the patient themself). In CPM mode in block 516 and volitional movement mode in block 518, the electrical signals that control movement of the orthosis device are externally induced (i.e., instructions generated by the control system of the orthosis device, not by the patient themself). Brain signals generated by the movement of the orthosis device are then captured by the headset, as indicated by the upward arrows between blocks 516/518 and block 512. The three modes available in the BCI-based apparatus of the present disclosure advantageously provide more detailed information compared to conventional orthosis systems, and this information can consequently be used to provide more complete assessments of rehabilitation outcomes than could otherwise be performed.

In some embodiments, block 510 can be an initial session or set of sessions to screen for compatibility of the subject with the BCI apparatus. In other embodiments, block 510 can be rehabilitation session during ongoing therapy of the patient. In either situation, brain signals and performance data from the orthosis device are collected.

In block 520, inputs are gathered for analysis. Inputs include data from the BCI-based apparatus, including brain signals 522 and orthosis measurements 523 from the orthosis device. Additional inputs involve one or more of the following: the patient's background information 524, which can include the patient's general information 525 and medical history 526; information from a broader database 527 of a general stroke population; and other assessments 528 of the patient. The present methods and systems uniquely utilize data from the orthosis device as well as background information about the patient to provide a holistic and more complete analysis of the patient's prognosis than conventional techniques can provide. The data provided by the orthosis device includes force and movement data, which are not typically available on all orthosis devices. Embodiments may also include data from other modalities such as cognitive tests, brain signals, and imaging assessments, which can further enhance the analysis of the human subject's rehabilitative outcome.

The brain signals 522 can include, but are not limited to, measurements of correlation, coherence, phase, amplitude, frequencies, amplitude disparity between hemispheres, baseline physiology. These brain signals can provide correlations of the patient's brain control signals with actions performed by the subject. For example, certain frequencies can be indicative of particular movements, and coherence can assess normality or abnormality of functional brain connectivity. In another example, measurements of gamma and theta brain waves from the patient can be used in the predictive analysis. Correlation between gamma and theta waves can help predict the likelihood of successful rehabilitation after stroke, where theta-gamma coupling increases as patients functionally improve over time. Methods of the present disclosure involve analyzing the various brain signal measurements along with other input data of block 520 to understand relationships between the factors to predict how the subject will respond to rehabilitation therapies. In embodiments, machine learning algorithms can be trained to understand how certain brain signal measurements associate more significantly with particular outcomes, such as a patient's compatibility with a BCI-based system or a patient's rehabilitation rate using a certain therapy mode. Machine learning can also be used to derive relationships between various brain signal measurements and how they correlate to predictive outcomes, either for that individual patient or as a general trend in stroke patients.

Orthosis measurements 523 can include data from various sensors of the orthosis device. Movement data can include extension, flexion, range of movement, and kinetics of a body part during a therapy session. Force data can be measured, for example, in response to volitional movement (e.g., measuring a resistance to movement), passive hand opening (to reflect spasticity), active grip strength, and/or extension force. For example, the force sensors annotated in FIGS. 2A-2D can include a load cell force sensor that is capable of sensing both a tension force and a compression force exerted on the sensor by push-pull wires in the orthosis device. Force sensors can also include force sensing resistors that measure forces when the sensors come in contact with each other, thus indicating an amount of movement and forces involved in the movement. Some embodiments may also include force measurements to perform the grip strength (grasp) assessments of motor tests such as a Fugl-Meyer Assessment for Upper Extremity (FMA-UE). For example, the orthosis device may be configured to have its force sensors measure flexion and extension against a prescribed resistance, and these forces may be used to derive the grasp evaluations. In further examples, force sensors in the orthosis device may be used to measure the flexion and extension forces of the patient's fingers when holding or pulling on a particular object, or when trying to resist a movement actuated by the orthosis device. In some embodiments, the movement sensors of the orthosis device can include an accelerometer and/or a gyroscope, where the movement data includes acceleration data from the accelerometer or gyroscope data from the gyroscope. Positional and kinematic data can be gathered by accelerometer measurements (acceleration of linear motion) and/or gyroscope measurements (angular velocity to derive tilt and lateral orientation).

Background information 524 of the subject can include general information 525 and medical history 526. A patient's general information 525 can include demographics (e.g., age, sex, and ethnicity) and socio-economic information (e.g., access to resources, income level, occupation, education). A patient's medical history 526 (i.e., medical history of the subject) can include stroke lesion characteristics such as lesion location, volume of infarct/extent of stroke, length of time from the occurrence of the stroke, diffusion tensor imaging (DTI), white matter tractography, functional network, organization and perturbation. The patient's medical history 526 can also include biometrics of the subject such as genetics, gut biome, medications the patient is taking, and comorbidities (e.g., depression, irritable bowel syndrome). By including background information of the patient in the stroke assessment, a comprehensive view of the patient's situation is provided, consequently enabling more accurate assessment of the patient's outcome. The predictive analysis can derive complex correlations that would be unable to be performed manually.

Broader database 527 can be information compiled for a general population of stroke patients. The database can represent, for example, a national or regional population, and can be gathered from information collected by professional and/or government organizations. The database 527 can include information for each stroke patient similar to those in block 520 such as demographics and lesion characteristics, along with other information related to rehabilitation paths such as types of therapies utilized, length of time undergoing therapy, and level of function regained after therapy. The broader database 527 can also include information on the patient being analyzed by the method 500, where the patient's information can be updated in the database 527 on an ongoing basis as indicated by arrow 535 during the patient's rehabilitation program.

Other assessments 528 are test results from sources other than the BCI-based system being used in block 510. These other assessments 528 can include imagery assessments of the subject such as computed tomography (CT) scans, magnetic resonance imaging (MRI), functional MRIs (fMRI), and anatomic imaging. These other assessments 528 can also include functional assessments performed by, for example, a physical therapist, occupational therapist or neurologist and can include non-BCI functional assessments such as one or more of: Fugl-Meyer, Ashworth spasticity, modified Ashworth, action research arm test (ARAT), arm motor ability test (AMAT), stroke impact scale (SIS), movement grades, gait analysis, box & blocks test, or motricity index. In embodiments, the input data used in systems and methods of the present disclosure includes these imagery assessments of the subject and non-BCI functional assessments. Information from these assessments 528 can be input or compiled into the cloud network by, for example, manual entry and/or by linking of medical care records from different entities associated with the patient's care. These various assessments 528 provide a more comprehensive view of the patient's stroke condition and overall medical status, which can be used to inform the predictive analysis of method 500 in a more holistic manner.

The input data may be prepared in block 532 to, for example, put the data from block 520 into a form for processing in block 530 and/or compile certain data factors in order to simplify or streamline calculations during block 530. In one embodiment, the data preparation of block 532 may involve converting qualitative data into numerical values or scores, such as assigning numerical values for socio-economic or demographic factors, or setting numerical codes for results from imaging assessments. In another embodiment, the data processing may include scaling or converting quantitative data into standardized units, such as for the force data, movement data, and brain signals. In another embodiment, the data processing may include aggregating groups of information for processing, such as synthesizing a single numerical value from the person's infarct size, infarct location, and length of time from the stroke to represent an overall severity of injury. One or more of these example data preparation steps may be used in any combination with each other.

Block 530 involves processing the input data from block 520 using instructions stored on a computer processor, where the instructions may include machine learning. The machine learning can involve any algorithm that processes the inputs from block 520 and calculates outputs in block 540. The instructions for the computer processor may be stored as non-transitory computer readable medium on a computer, where the instructions cause the computer to perform methods for assessing a stroke rehabilitation outcome of a subject as described herein. The algorithms may involve supervised learning, unsupervised learning, or semi-supervised learning to model relationships between the input data of block 520 and predict outcomes. In some embodiments, supervised learning is used where the algorithm can be support vector machines, nearest neighbor, Naive Bayes, linear regression, or neural networks. In one specific example, an LT/ST recursive neural network may be used, utilizing time traces for inputs such as force kinematics, and scalars with long EEG traces. In some embodiments, the algorithm can weight particular data or determine what input data to use based on an individual's profile such as their type of injury, baseline physiology, ongoing physiology, amount of time after stroke event, and the like.

In some embodiments, weighting factors may be applied to one or more data in the input data. The instructions for the computer processor may include the application and calculation of these weighting factors to particular input data based on, for example, the values of the input data or the individual patient's profile that may indicate that certain data factors may play a stronger role in their outcomes than other factors. The use of weighting factors (i.e., coefficients) for specific input data and the value assigned to those weighting factors may depend on where the patient is in the distribution relative to the overall stroke population (broader database 527). That is, the computer processing instructions (e.g., a machine learning algorithm) to process the input data may comprise determining and applying a weighting factor that is based on a ranking of where a data factor of the input data (i.e., a specific type of data in input block 520, such as the brain signal, the force data, the movement data, or the background information) lies within the broader stroke population database. In embodiments, the instructions for the computer processor to process the input data comprise determining and applying (i.e., application of) a weighting factor based on a ranking of where the brain signal, the force data, the movement data, or the background information lies within a broader stroke population database. For example, if the patient's degree of infarct (or other data factor) is in a nominal range of the stroke population, the weighting coefficient for that lesion characteristic may be assigned a small or zero value. If the subject's degree of infarct (or other input factor) is more out of range from the average stroke population, that data factor may be assigned higher weighting coefficient value. In embodiments, the instructions to process the input data comprise applying a weighting factor based on a severity level of the stroke lesion characteristics. In some embodiments, the instructions to process the input data comprise calculating a weighting factor for one or more of the input data based on the background information. For instance, the subject's demographics (e.g., age, ethnicity) and socio-economic information (e.g., access to resources, occupation) may help indicate the subject's likely rate of rehabilitation or their propensity for successful rehabilitation. As a specific example, a subject who is younger and has more resources than an average stroke patient may have higher rehabilitation rates, and these data factors can be weighted accordingly by the predictive assessment.

In some embodiments, the data processing of block 530 can determine weighting factors based on an absolute value of a data factor for that individual alone. For example, the absence or strong presence of a particular brain signal measurement (e.g., certain brain wave signal) may be used to train the machine learning algorithm on how that signal impacts the patient's rehabilitation rate or the ability to recover a particular motor movement. In embodiments, the instructions to process the input data comprise applying a weighting factor based on a strength of the brain signal. Similarly, the severity of an underlying medical condition can be correlated with particular therapies that may be more helpful for that patient. In some embodiments, trends between input data can be identified by the machine learning algorithm that seem disparate but correlate to improved rehabilitation. The correlations and trends identified by the computer processor may be made possible by the specialized computer software and algorithms, beyond what can be achieved by manual calculations.

In some embodiments, the data processing of block 530 can be trained to predict the individual's rehabilitation trajectory, to optimize the patient's treatment based on the broader database 527 of the larger population, to improve the BCI interface for the individual patient (i.e., learn how to improve brain signal correlation for that patient as the patient progresses), or to incorporate brain lesion mapping into the algorithm.

The calculations of block 530 results in assessments of the subject's stroke rehabilitation being output in block 540. These outputs can include a prediction of rehabilitation outcome 542 and/or categorization of a patient's rehabilitation status 544. The analysis can produce, for example, a “stroke mass index” or other composite score that can be used to inform decisions on a personalized rehabilitation program for the patient. The composite score may be, for example, a numerical value (e.g., scale of 1 to 10 or percentile ranking from 0% to 100% to assess their likelihood of full rehabilitation) or a qualitative ranking (e.g., below average, average, very good, excellent). The composite score takes into consideration the various input data from the patient such as brain signals gathered from the brain signal acquisition headset, force data and movement data gathered by the orthosis device, and background information about the subject. As an example, the background information can include one or more of the patient's medical condition information (e.g., infarct size, infarct location, physiology), socio-economic information and demographics. The computer processor can analyze the input data and determine the composite score based on, for example, outcomes from a broader population of stroke patients with similar characteristics, and/or from history of the individual subject's progress.

In some embodiments of rehabilitation outcome 542, the rehabilitation outcome prediction can be used as a screening tool to estimate compatibility of the subject with the BCI apparatus. For example, the ability of the subject to interface with the BCI apparatus, or the ability to control certain motions, can be predicted. In some embodiments of rehabilitation outcome 542, the output rehabilitation outcome prediction can be a prediction of rehabilitation response rate of the subject using the BCI apparatus. The rehabilitation response rate may be expressed, for example, as a percent improvement over a predetermined amount of time (e.g., 25% improvement in Fugl-Meyer score in 90 days). Another example of a rehabilitation response rate is a prediction of the total amount of time or total number of sessions that the patient will need to use the BCI apparatus to achieve a desired improvement level.

In some embodiments, the system processes the input data for outputting a categorization of a rehabilitation status 544 of the subject. Categorizing a patient's rehabilitation status 544 can serve as a tracking tool to monitor the patient's progress during rehabilitation. For example, the categorization can be quantified in terms of a percentile or ranking relative to the general stroke population, or as an amount of improvement relative to the patient's starting point. In some embodiments, this tracking or monitoring can be qualitative in addition to or instead of quantitative metrics, where the qualitative measures can be, for example, how the patient feels (e.g., mood, pain). The categorization of a rehabilitation status 544 can serve as a motivational tool for the subject in which they can view their improvement over time. Thus, the rehabilitation status 544 serves as a functional biomarker/biometric to assess the patient's rehabilitation progress, such as relative to a general distribution of stroke patients. The categorization of a rehabilitation status 544 can be used as a biomarker for recovery and/or to track improvement, being an adaptive tool throughout the patient's rehabilitation.

In further embodiments of outputs in block 540 that can be produced by the method 500, the algorithm can automatically generate recommendations for therapy based on, for example, similar patient cohorts or the patient's current progress. In embodiments, instructions for the computer processor include outputting a modification to a rehabilitation program used by the subject, based on the rehabilitation outcome prediction. The recommendations can be generated without input from a clinician, although the subject may choose to consult with a clinician before changing their therapy practices. In some embodiments, the computer processing instructions may include outputting (e.g., using machine learning) a revised rehabilitation program compared to what the subject was using before. For example, the revised rehabilitation program can add, remove, or alter particular exercises from the patient's rehabilitation plan to target certain movements that are progressing slower or faster than expected. Examples of altering exercises may include, for example, changing the number of repetitions, modifying a range of motion, or substituting an exercise with another one that may be more effective for that particular patient, Therapy optimization may also include outputting information on alternative options instead of or in addition to BCI-based therapy, such as medication (e.g., BOTOX® or other enhancing agents for improving central plasticity or to address spasticity), physical therapy, vibratory stimulation, electrical stimulation, or other rehabilitative tools. Other therapies that the computer process may output as recommendations to optimize therapy may include wellness and lifestyle techniques such as yoga, meditation, and dietary changes. Further embodiments may optimize outcome for a given cost threshold, such as to achieve a desired amount of improvement within constraints imposed by insurance coverage and/or the individual's financial status.

In embodiments of the method 500, after data is processed in block 530 the patient's rehabilitation can continued to be monitored in block 550 by performing another therapy session in block 510. This cycle of performing a therapy session in block 510, gathering input data in block 520, processing data in block 530, and monitoring rehabilitation progress in block 550 can then be repeated, with outputs in block 540 being updated if desired after each cycle. In each cycle, the data processing in block 530 can be adapted to account for updated input data of block 520, and the rehabilitation progress can be added to the broader stroke population database 527. For example, the method can involve repeating the collecting to collect updated brain signals, force data and movement data from the home-based BCI apparatus, and revising calculation parameters for the computer processing instructions (e.g., machine learning algorithm) using the updated brain signals, force data and movement data.

In an example method of method 500, a method for assessing a stroke rehabilitation outcome of a subject includes collecting a brain signal, force data and movement data from a home-based brain-controlled interface (BCI) apparatus. The method also includes processing input data, using a computer processor. The input data comprises background information about the subject along with the brain signal, the force data and the movement data to output a rehabilitation outcome prediction for the subject. The home-based BCI apparatus includes: i) a portable brain signal acquisition headset that acquires the brain signal from a subject, ii) an orthosis device, iii) a BCI component that receives the brain signal from the brain signal acquisition headset and that is capable of controlling of the body part interface, and iv) a control system. The orthosis device includes a body part interface configured to be coupled to a body part of the subject. The orthosis device also includes a plurality of sensors that generate the force data and the movement data. In some embodiments, the orthosis device includes a motor-actuated assembly that actuates the body part interface to move the body part. The plurality of sensors may include a force sensor that measures force data applied between body part interface and the motor-actuated assembly, and a movement sensor that measures movement data of the body part interface. The control system is configured to operate the orthosis device in three modes: a BCI mode in which the orthosis device operates to move or assist in the movement of the body part based on an intention of the subject determined from an analysis of the brain signal; a continuous passive mode in which the orthosis device operates to move the body part with no volition from the subject; and a volitional mode in which the orthosis device first allows the subject to move or attempt to move the impaired body part in a predefined motion and then operates to move or assist in the predefined motion of the body part.

FIG. 5B is a flowchart of a method 501 for training a machine learning algorithm for assessing a stroke rehabilitation outcome of a subject, in accordance with some embodiments. Description of blocks in FIG. 5A apply to those blocks in FIG. 5B with the same reference numbers. Input data is collected in block 520 to train the machine learning algorithm. The input data may include historical data gathered from a sample patient as described in relation to FIG. 5A and/or gathered from a broader database 527 (e.g., compiled from subjects at a particular facility and/or other databases). The data is prepared in block 532, which may involve, for example, converting qualitative data into numerical values or scores, scaling or converting quantitative data into standardized units, or aggregating groups of information for processing. Data processing is performed in block 530 by a computer processor as described in relation to FIG. 5A. For example, block 530 may involve determining which data of the input data are important to include in the calculations, applying weighting factors to one or more types of data in the input data, and calculating predicted outcomes for the patient. The data processing in block 530 may involve a computer processor that performs steps using machine learning algorithms.

Assessments or predictions of the subject's stroke rehabilitation are output in block 540 (e.g., predicting the rehabilitation outcome 542 and/or categorizing the patient's rehabilitation status 544) and are compared in block 574 to a known outcome for the patient and/or similar outcomes of similar patients. Based on a comparison of differences between the calculated output and the actual outcomes in block 574 (e.g., the comparison may be calculated losses between predicted and actual values), calculation parameters for the computer processor instructions are revised in block 576. For example, weighting factors, relationships between various input data, and correlation coefficients or equations used in the data processing of block 530 can be adjusted to more accurately match the known outcomes. The calculation parameters in block 576 are then updated for the data processing of block 530. Embodiments of method 501 include loop 578 of repeating the collecting of data to collect updated brain signals, updated force data and updated movement data from the home-based BCI apparatus, and revising instructions for processing the input data based on the updated brain signals, the updated force data, and the updated movement data. The training as described by method 501 may be repeated as more input data in block 520 is provided over time.

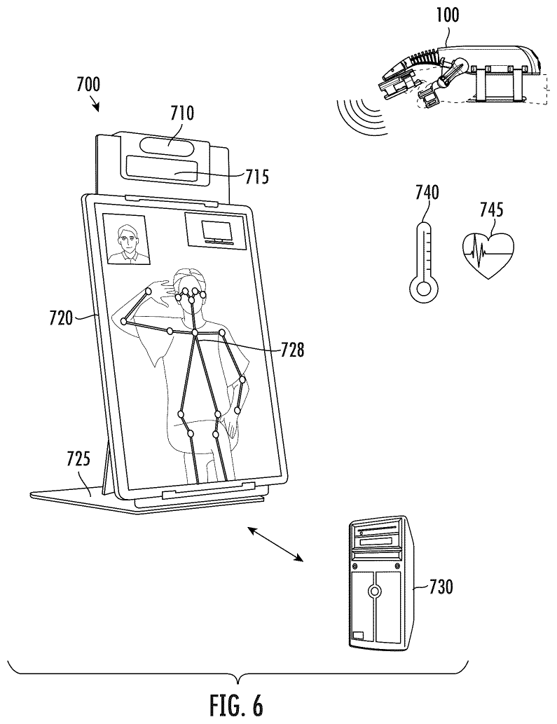

In some embodiments, the home-based assessment system of the present disclosure can include camera hardware and customized skeleton tracking software as shown in FIG. 6 to extend the type of measurements available to be input in block 520. FIG. 6 illustrates an example system 700 that includes a depth camera 710, a tracking camera 715, and a computing device 720 on an optional stand 725. The equipment of FIG. 6 can be used, for example, for remote assessment of motor function (e.g., upper or lower extremity function) in addition to those that can be obtained from the orthosis device. In FIG. 6 the computing device 720 is illustrated as a tablet computer. In other embodiments, the computing device 720 can be, for example, a mobile phone, laptop computer or a personal computer. The system 700 may be configured to communicate with the mobile device (computing device 720), and the mobile device displays directions on custom user interface 728 for performing the movement. The computing device 720 communicates with the orthosis device 100 and may also be connected to a central processor 730 such as a cloud computing system. Additional information from sensors such as environmental sensors 740 (e.g., for room temperature, ambient light level or other) and biometric sensors 745 (e.g., for heart rate, oxygen level or other) may also be supplied to the computing device 720 and the central processor 730 for use in the analysis of the subject's rehabilitation session. In embodiments, the system 700 includes an environmental sensor or a biometric sensor, wherein the input data includes data from the environmental sensor or the biometric sensor.

Camera hardware (depth camera 710 and/or tracking camera 715) may be stereoscopic or non-stereoscopic. Example technology that may be utilized for the application and implementation of the virtual motor assessment may include, but are not limited to, INTEL® RealSense Skeleton Tracking SDK with Depth Mapping (by Cubemos), Intel RealSense Hand Tracking, IpsiHand System tablet computer from Neurolutions (Santa Cruz, CA), Neurolutions Integrated Tablet Stand, and Neurolutions IpsiHand orthosis device (e.g., device 100).

In an example embodiment, the Intel RealSense Skeleton Tracking SDK with Depth Mapping employs cameras that use active infrared stereo vision to provide accurate depth and position of objects within its field of view, while the SDKs use this data to build a skeleton model with 18 joints within the body frame in 2D/3D. This camera (or similar cameras) allows the measurement and comparison of specific joint angles and upper/lower extremity rotation when completing specific and active movements of the upper/lower extremities. Conventionally, these measurements and comparisons are completed by visual observation of the assessor. Implementation of skeleton tracking with depth mapping allows the addition of precision and accuracy to visual observation assessment by the clinician. For example, the Intel RealSense Hand Tracking allows the joints and bones of the hands to be tracked using 22 points of tracking. Motion tracking software used in embodiments of the present disclosure can assess hand movements as directed when completing motor assessments. The tablet stand is designed to position the cameras and the user interface—such as the 15″ IpsiHand touchscreen tablet personal computer (PC) and the depth cameras (e.g., Intel D435 or D455) and tracking cameras (e.g., Intel T265)—at a comfortable angle for seated assessment. The cameras may connect to the tablet PC via, for example, USB ports.

Referring again to FIG. 5A, in some embodiments, the input data of block 520 can include information from other sources such as wearable monitors (e.g., heart rate or oxygen sensors on smartphones or smart bracelets/rings). Other sensors that may be used in addition to those mentioned elsewhere in this disclosure include, but are not limited to, electrical current sensors, electromyography sensors, electrocardiograms, temperature sensors, and biometric sensors such as pulse/heart rate, oximeter, and stress/perspiration (e.g., analyte/molecular sensors for specific biological substances). Data from the various sensors can be used, for example, to derive the amount of effort required by the patient for particular movements, or to determine environmental conditions (e.g., temperature) that may impact the patient's performance during an evaluation. Any of the sensors in this disclosure may be used alone or in combination with each other to assess information about the patient's rehabilitation progress, customize therapy plans, and predict outcomes for the patient. In some embodiments, measurements from these sensors as well as interactions between the measurements (e.g., correlation, coherence) can be used as input data to predict rehabilitation outcomes or to categorize a patient's rehabilitation status in accordance with the present disclosure.

In further embodiments, data from force sensors of the orthosis device may be used to measure motor evoked potentials (MEP), which can then be further used to help assess rehabilitation outcomes for the patient. In such embodiments, the system can uniquely measure a somato-sensory evoked potential (SSEP), in which brain signals are measured as a result of BCI motor-generated movements. That is, the home-based system 400 can be used to interrogate the subject's motor system, where the force sensors of the orthosis device are used to represent the presence of an MEP signal. In such embodiments, a motor mechanism of an orthosis device is actuated to cause passive movement, during which force signals are generated by the force sensors of the orthosis device. Brain signals stimulated by the movement are recorded, which in effect creates a “reverse MEP.” In some embodiments, multiple actuations may be performed to result in a multitude of high-frequency sensor inputs that are time-locked, with a short rest interval in between. An average of the time series may be taken to create the SSEP. For the actuations, full hand closures may be utilized, or rapid twitches may be sufficient (i.e., small movements may be enough to generate a brain signal). The SSEP measurements provide an indication of the integrity of cortico-spinal tract, similar to conventional MEPs but in the opposite direction (brain signals stimulated by motor movement, rather than brain signals causing electrical stimulation in muscles as in MEPs). These orthoses-generated SSEPs as described herein can be utilized in the system and method of FIGS. 4 and 5A-5B, such as to see if the SSEPs change over time during a rehabilitation program. Performing such an SSEP or “reverse MEP” provides an empirical measure of whether stimulation can be measured at the peripheral component to invoke a brain response. If the patient exhibits a good MEP, then this result can be used in the predictive analysis to indicate that the patient is likely to be compatible with a BCI-based rehabilitation device or other types of tools. In embodiments, the input data comprises a somato-sensory evoked potential, wherein the computer processor determines the SSEP from the brain signal acquired as a result of a BCI motor-generated movement from the continuous passive mode and the force data measured by a force sensor in the plurality of sensors during the BCI motor-generated movement. Embodiments also include methods involving determining the SSEP, where the SSEP is used as part of the input data.

The systems and methods of the present disclosure utilize new combinations of input data to assess a subject's rehabilitation outcome. The input data may include one or more of the data factors described herein, such as functional performance measurements by a BCI apparatus (e.g., force data, movement data, brain signals), background information about the subject (e.g., medical history, demographics, socio-economic information), non-BCI assessments (e.g., imaging tests) and other data from therapy sessions (e.g., environmental and/or biometric information). This extent of the types of data used for stroke rehabilitation assessments has not been used in this field and presents a complex analysis that is not intuitive. The outputs from the present systems and methods—such as likelihood of success, categorization of the individual's rehabilitation status, and therapy modifications—also provide unique benefits over standard methods. For example, the rehabilitation assessments are more individualized and comprehensive than can be achieved with conventional techniques, and thus can provide more motivation for patients to perform their therapy programs and with better results. These results can be utilized by more patients due to the home-based nature of the systems and methods, enabling improved ease of use and access.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A system for assessing a stroke rehabilitation outcome of a subject, the system comprising:
a) a home-based brain-controlled interface (BCI) apparatus having:
i) a portable brain signal acquisition headset that acquires a brain signal from the subject;
ii) an orthosis device having a body part interface configured to be coupled to a body part of the subject, and having a plurality of sensors that generate force data and movement data;
iii) a BCI component that receives the brain signal from the brain signal acquisition headset, wherein the BCI component is capable of controlling the orthosis device; and
b) a computer processor in communication with the BCI apparatus, the computer processor performing instructions to process input data to output a rehabilitation outcome prediction for the subject, wherein the input data includes the brain signal, the force data, the movement data, and background information about the subject.

2. The system of claim 1, wherein the BCI apparatus further comprises:
iv) a control system configured to operate the orthosis device in:
a BCI mode in which the orthosis device operates to move or assist in a movement of the body part based on an intention of the subject determined from an analysis of the brain signal;
a continuous passive mode in which the orthosis device operates to move the body part with no volition from the subject; and
a volitional mode in which the orthosis device first allows the subject to move or attempt to move the body part in a predefined motion and then operates to move or assist in the predefined motion of the body part.

3. The system of claim 2, wherein the input data comprises a somato-sensory evoked potential (SSEP), wherein the computer processor determines the SSEP from:
the brain signal acquired as a result of a BCI motor-generated movement from the continuous passive mode; and
the force data measured by a force sensor in the plurality of sensors during the BCI motor-generated movement.

4. The system of claim 1, wherein:
the plurality of sensors comprises an accelerometer or a gyroscope; and
the movement data includes acceleration data from the accelerometer or gyroscope data from the gyroscope.

5. The system of claim 1, wherein:
the orthosis device comprises a motor-actuated assembly that actuates the body part interface to move the body part;
the plurality of sensors comprises a force sensor that generates the force data; and
the force data includes forces applied between the body part interface and the motor-actuated assembly.

6. The system of claim 1, wherein the background information includes demographics and socio-economic information of the subject.

7. The system of claim 1, wherein the background information includes a medical history of the subject, the medical history including stroke lesion characteristics and biometrics.

8. The system of claim 7, wherein the instructions to process the input data comprise application of a weighting factor based on a severity level of the stroke lesion characteristics.

9. The system of claim 1, wherein the instructions to process the input data comprise calculation of a weighting factor for one or more of the input data based on the background information.

10. The system of claim 1, wherein the instructions to process the input data comprise application of a weighting factor based on a strength of the brain signal.

11. The system of claim 1, wherein the instructions to process the input data comprise application of a weighting factor based on a ranking of where the brain signal, the force data, the movement data, or the background information lies within a broader stroke population database.

12. The system of claim 1, further comprising an environmental sensor or a biometric sensor, wherein the input data includes data from the environmental sensor or the biometric sensor.

13. The system of claim 1, wherein the input data further includes imagery assessments of the subject and non-BCI functional assessments.

14. The system of claim 1, wherein the rehabilitation outcome prediction comprises a compatibility of the subject with the BCI apparatus, or a rehabilitation response rate of the subject using the BCI apparatus.

15. The system of claim 1, wherein the instructions comprise outputting a modification to a rehabilitation program used by the subject, based on the rehabilitation outcome prediction.

16. The system of claim 1, wherein the instructions comprise outputting a categorization of a rehabilitation status of the subject.

17. A method for assessing a stroke rehabilitation outcome of a subject, the method comprising:
a) collecting a brain signal, force data and movement data from a home-based brain-controlled interface (BCI) apparatus; and
b) processing input data, using a computer processor that performs instructions for the processing, wherein the input data comprises background information about the subject along with the brain signal, the force data and the movement data to output a rehabilitation outcome prediction for the subject;
wherein the home-based BCI apparatus includes:
i) a portable brain signal acquisition headset that acquires the brain signal;
ii) an orthosis device having a body part interface configured to be coupled to a body part of the subject, and having a plurality of sensors that generate the force data and the movement data; and
iii) a BCI component that receives the brain signal from the brain signal acquisition headset, wherein the BCI component is capable of controlling of the orthosis device.

18. The method of claim 17, wherein the BCI apparatus further comprises:
iv) a control system configured to operate the orthosis device in:
a BCI mode in which the orthosis device operates to move or assist in a movement of the body part based on an intention of the subject determined from an analysis of the brain signal;
a continuous passive mode in which the orthosis device operates to move the body part with no volition from the subject; and
a volitional mode in which the orthosis device first allows the subject to move or attempt to move the body part in a predefined motion and then operates to move or assist in the predefined motion of the body part.

19. The method of claim 18, further comprising determining a somato-sensory evoked potential (SSEP), wherein the input data comprises the SSEP, and wherein determining the SSEP comprises:
measuring the brain signal as a result of a BCI motor-generated movement in the continuous passive mode; and
using a force sensor in the plurality of sensors to measure the force data during the BCI motor-generated movement.

20. The method of claim 17, wherein the background information includes demographics and socio-economic information of the subject.

21. The method of claim 17, wherein the background information includes a medical history of the subject, the medical history including stroke lesion characteristics and biometrics.

22. The method of claim 21, wherein processing the input data comprises applying a weighting factor based on a severity level of the stroke lesion characteristics.

23. The method of claim 17, wherein processing the input data comprises calculating a weighting factor for one or more of the input data based on the background information.

24. The method of claim 17, wherein the instructions to process the input data comprise applying a weighting factor based on a strength of the brain signal.

25. The method of claim 17, further comprising determining a weighting factor based on a ranking of where the brain signal, the force data, the movement data, or the background information lies within a broader stroke population database.

26. The method of claim 17, wherein the input data further includes imagery assessments of the subject and non-BCI functional assessments.

27. The method of claim 17, wherein the rehabilitation outcome prediction comprises a compatibility of the subject with the BCI apparatus, or a rehabilitation response rate of the subject using the BCI apparatus.

28. The method of claim 17, further comprising outputting a modification to a rehabilitation program used by the subject, based on the rehabilitation outcome prediction.

29. The method of claim 17, further comprising outputting a categorization of a rehabilitation status of the subject.

30. The method of claim 17, further comprising:
repeating the collecting to collect updated brain signals, updated force data and updated movement data from the home-based BCI apparatus; and
revising instructions for processing the input data based on the updated brain signals, the updated force data and the updated movement data.
